(19) United States
(12) Patent Application Publication
(10) Pub. No.: US 2026/0215963 A1
(43) Pub. Date: Jul. 30, 2026

(54) TREATMENT OF OCULAR INJURIES USING MICROBOTS

(71) Applicant: Applied Research Associates, Inc., Albuquerque, NM (US)

(72) Inventor: Louis William Rogowski, Randolph, VT (US)

(21) Appl. No.: 19/459,690

(22) Filed: Jan. 26, 2026

Related U.S. Application Data
(60) Provisional application No. 63/749,914, filed on Jan. 27, 2025.

(51) Int. Cl. A61F 9/00; A61B 34/30; A61K 38/18; A61K 38/48; A61K 38/49

(52) U.S. Cl. CPC A61F 9/0026; A61B 34/30; A61K 38/185; A61K 38/482; A61K 38/49

(57) ABSTRACT

Systems, methods, and computer-readable media for delivering drugs to an ocular region. A system for delivering drugs may include a microbot, the microbot comprising a biocompatible body, a plurality of magnetic particles, and a drug payload. The microbot may be configured to be navigated within a biofluid to a target ocular region via a magnetic control system. The microbot may be configured to automatically release the drug payload into a surrounding environment of the target ocular region in response to a condition being met. The drug payload may comprise a thrombolytic compound configured to degrade coagulated blood upon the release of the drug payload into the surrounding environment. Further, the drug payload may comprise molecules configured to promote survivability of retinal ganglion cells (RGCs) upon the release of the drug payload into the surrounding environment.

### CROSS-REFERENCE TO RELATED APPLICATIONS

This patent application is a non-provisional application claiming priority benefit, with regard to all common subject matter, of U.S. Provisional Patent Application No. 63/749,914, filed Jan. 27, 2025, and entitled “TREATMENT OF OCULAR INJURIES USING MICROBOTS.” The above-referenced application is hereby incorporated by reference in its entirety into the present application.

### BACKGROUND

### 1. Field

Embodiments of the present disclosure relate to systems for delivering payloads using microbots. Specifically, embodiments of the present disclosure relate to systems for delivering payloads to an ocular region using microbots and a magnetic control system.

### 2. Related Art

Ocular trauma remains a significant challenge for individuals experiencing various types of accidents or injuries, with two pressing issues being vitreous hemorrhage and retinal ganglion cell (RGC) loss. These injuries are prevalent across various populations because of trauma or damage to an individual's eye. Vitreous hemorrhages cause temporary vision loss, while RGC loss can permanently damage a person's vision. Both conditions impair an individual's quality of life and ability to perform daily activities. Addressing these issues requires innovative solutions to improve recovery times and prevent long-term damage.

Vitreous hemorrhage is a common ocular injury that occurs when blood vessels in the eye rupture, causing blood to pool between the retina and other critical structures. The blood that collects in the vitreous humor forms clots, or “floaters,” which obstruct vision. In extreme cases, large clots can cause prolonged total vision loss lasting for months. Natural recovery is slow, as the eye clears the blood at a rate of about 1% per day, leading to a recovery period of up to four months. Surgical interventions, such as vitrectomy, involve removing the vitreous fluid and replacing it with a saline solution to speed up recovery. However, these surgeries have risks such as increased ocular pressure, infections, retinal detachment, and other complications. The lengthy recovery times and potential side effects pose a significant burden to affected individuals, making vitreous hemorrhage a pressing issue in civilian healthcare as well as in military settings, where such injuries can occur in blast-related incidents.

RGCs play a role in transmitting visual information from the retina to the brain. These cells are highly susceptible to damage from trauma, including traumatic brain injuries, blunt impacts, or exposure to harmful substances. RGC loss can occur from direct damage to the optic nerve or secondary effects like ischemia or retinal detachment. Unlike vitreous hemorrhages, RGC damage is often irreversible, leading to permanent vision loss. Current research has explored the possibility of transplanting RGCs, but challenges remain in ensuring transplanted cells' survival and re-establishing axonal connections with the optic nerve. Without effective interventions, RGC loss continues to be a critical issue for individuals exposed to situations that risk ocular and neurological damage.

Both vitreous hemorrhage and RGC loss highlight the urgent need for new treatment solutions to address ocular injuries more effectively. While vitreous hemorrhages can be treated over time or through surgery, the lengthy recovery periods hinder an individual's ability to return to normal activities promptly. Meanwhile, the irreversible nature of RGC damage underscores the necessity of developing preventative or regenerative therapies to protect neurosensory functions. In both cases, advancements in medical technologies and rehabilitation strategies could significantly improve recovery outcomes and quality of life for injured individuals.

### SUMMARY

Embodiments of the present disclosure solve the problems mentioned above by providing treatment options using microbots to deliver drug payloads to injured regions of the eye.

In some embodiments, the techniques described herein relate to a microbot configured for delivering drugs to an ocular region, the microbot including: a biocompatible body; a plurality of magnetic particles; and a drug payload, wherein the microbot is configured to be navigated within a biofluid to a target ocular region via a magnetic control system, wherein the microbot is configured to automatically release the drug payload into a surrounding environment of the target ocular region in response to a condition being met.

In some embodiments, the techniques described herein relate to a microbot, wherein the drug payload includes a thrombolytic compound configured to degrade coagulated blood upon the release of the drug payload into the surrounding environment.

In some embodiments, the techniques described herein relate to a microbot, wherein the thrombolytic compound at least includes tissue plasminogen activator (tPA) or streptokinase.

In some embodiments, the techniques described herein relate to a microbot, wherein the drug payload includes molecules configured to promote survivability of retinal ganglion cells (RGCs) upon the release of the drug payload into the surrounding environment.

In some embodiments, the techniques described herein relate to a microbot, wherein the molecules are at least brain-derived neurotrophic factor (BDNF) protein molecules.

In some embodiments, the techniques described herein relate to a microbot, wherein the condition is the surrounding environment reaching a predetermined temperature.

In some embodiments, the techniques described herein relate to a microbot, wherein the drug payload includes chemical additives configured to modulate a release rate of the release of the drug payload into the surrounding environment.

In some embodiments, the techniques described herein relate to a microbot, wherein the chemical additives are co-polymer additives, wherein the release of the drug payload occurs upon the co-polymer additives detecting the predetermined temperature of the surrounding environment.

In some embodiments, the techniques described herein relate to a microbot, wherein the release rate of the drug payload is a controlled rate such that the drug payload is uniformly released over a length of time upon the condition being met.

In some embodiments, the techniques described herein relate to a microbot, wherein the condition is the surrounding environment reaching a predetermined pH level.

In some embodiments, the techniques described herein relate to a microbot, wherein the plurality of magnetic particles and the drug payload are dispersed throughout the biocompatible body.

In some embodiments, the techniques described herein relate to a microbot, wherein the biocompatible body is an alginate hydrogel.

In some embodiments, the techniques described herein relate to a microbot, wherein the microbot has a width between 1 μm to 200 μm.

In some embodiments, the techniques described herein relate to a system configured for delivering drugs to an ocular region, including: a microbot, including: a biocompatible body; a plurality of magnetic particles; and a drug payload; and a magnetic control system, wherein the magnetic control system is configured to navigate the microbot within a biofluid to a target ocular region, wherein the microbot is configured to automatically release the drug payload into a surrounding environment of the target ocular region in response to a condition being met.

In some embodiments, the techniques described herein relate to a system, wherein the magnetic control system includes a plurality of Helmholtz coils, wherein selective rotation of the plurality of Helmholtz coils corresponds to selective movement of the microbot within the biofluid.

In some embodiments, the techniques described herein relate to a system, wherein the magnetic control system is configured to navigate the microbot within the biofluid along any of an x-axis, a y-axis, or a z-axis.

In some embodiments, the techniques described herein relate to a system, wherein the magnetic control system is configured to navigate the microbot to the target ocular region using closed-loop feedback.

In some embodiments, the techniques described herein relate to a system, wherein the target ocular region is a location within a vitreous humor region.

In some embodiments, the techniques described herein relate to a system, further including: a second microbot, including: a second biocompatible body; a second plurality of magnetic particles; and a second drug payload; and wherein the magnetic control system is configured to navigate the second microbot within the biofluid to the target ocular region, wherein the second microbot is configured to automatically release the second drug payload into the surrounding environment of the target ocular region in response to the condition being met.

In some embodiments, the techniques described herein relate to a method for delivering drugs to an ocular region, the method including: administering a plurality of microbots into an ocular component, each of the plurality of microbots including: a biocompatible body; a plurality of magnetic particles; and a drug payload; controlling the plurality of microbots via a magnetic control system, wherein the magnetic control system is configured to navigate the plurality of microbots within a biofluid to a target ocular region; and releasing, automatically in response to a condition being met, the drug payload into a surrounding environment of the target ocular region.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the present disclosure will be apparent from the following detailed description of the embodiments and the accompanying drawing figures.

### BRIEF DESCRIPTION OF THE DRAWING FIGURES

Embodiments of the present disclosure are described in detail below with reference to the attached drawing figures, wherein:

FIG. 1 illustrates an exemplary hardware platform in accordance with embodiments described herein.

FIG. 2 illustrates an exemplary drug delivery system in accordance with embodiments described herein.

FIG. 3 illustrates an exemplary fabrication method for fabricating microbots in accordance with described herein.

FIG. 4A illustrates an exemplary microbot used in accordance with embodiments described herein.

FIG. 4B illustrates an exemplary microbot used in accordance with embodiments described herein.

FIG. 5 illustrates an exemplary interface used in accordance with embodiments described herein.

FIG. 6A illustrates a first exemplary depiction of treating an ocular injury using microbots.

FIG. 6B illustrates a second exemplary depiction of treating an ocular injury using microbots.

FIG. 6C illustrates a third exemplary depiction of treating an ocular injury using microbots.

FIG. 7 illustrates an exemplary method for delivering drugs using microbots in accordance with embodiments described herein.

The drawing figures do not limit the present disclosure to the specific embodiments disclosed and described herein. The drawings are not necessarily to scale, emphasis instead being placed upon clearly illustrating the principles of the present disclosure.

### DETAILED DESCRIPTION

The following detailed description references the accompanying drawings that illustrate specific embodiments in which the present disclosure can be practiced. The embodiments are intended to describe aspects of the present disclosure in sufficient detail to enable those skilled in the art to practice the present disclosure. Other embodiments can be utilized and changes can be made without departing from the scope of the present disclosure. The following detailed description is, therefore, not to be taken in a limiting sense. The scope of the present disclosure is defined only by the appended claims, along with the full scope of equivalents to which such claims are entitled.

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the technology can include a variety of combinations and/or integrations of the embodiments described herein.

Embodiments of the present disclosure relate to a system for delivering payloads to ocular regions using magnetic navigation of microbots (e.g., artificial cell). For example, the microbots may be loaded (e.g., embedded, dispersed throughout, or encapsulated) with various molecules, such as drug compounds or proteins, and navigated to a target region using a magnetic control system. For instance, the microbots may be administered in vivo within a living organism such as a human or animal, where an external magnetic control system may be used to navigate the microbots within a biofluid so that the microbots reach the target region.

In some embodiments, the target region is a target ocular region (e.g., vitreous humor). For example, the microbots may be administered (e.g., injected) into an ocular component, such as the vitreous humor, where the magnetic control system may navigate the microbots to a target region within the vitreous humor of the eye. The microbots may be navigated within a biofluid of the vitreous humor to a region of the vitreous humor containing coagulated blood or a region experiencing retinal ganglion cell loss. Upon reaching the target ocular region, the microbots may automatically release the drug compounds and/or the different proteins.

For example, the microbots may comprise a drug payload. The drug payload may comprise a clot-dissolving thrombolytic compound configured to degrade (e.g., dissolve, break down, or destroy) coagulated blood when released into a surrounding environment of the target ocular region. For instance, a patient may have a vitreous hemorrhage injury where a vein has ruptured in the eye, causing coagulated blood to form (e.g., blood clotting) between the lens and the retina in the eye. To treat the vitreous hemorrhage, the thrombolytic compound released from the microbots may degrade or break down the coagulated blood's red blood cells. In some embodiments, the released drug payload breaks up the coagulated blood through hemolysis. Using a thrombolytic compound to treat a vitreous hemorrhage injury may allow a patient to recover from any visual impairment caused by the ruptured vein more quickly than the recovery times seen from natural recovery or after traditional surgical options have been performed. For instance, the coagulated blood may be degraded by the thrombolytic compound at an accelerated rate without requiring highly invasive vitrectomies or long natural recovery times. Instead of months or weeks, the patient may achieve a recovery time of several days. In another example, the drug payload comprises protein molecules (e.g., brain-derived neurotrophic factor (BDNF) protein molecules). The protein molecules may be configured to promote the survivability of RGCs upon the release of the drug payload into the surrounding environment of the target ocular region, helping to mitigate the loss of RGCs and prevent total vision loss of a patient.

Further, the drug payloads of the microbots may be released based on one or more conditions. For example, the thrombolytic compounds or the protein molecules contained within the microbots may be selectively released into the surrounding environment of the target ocular region at a predetermined temperature, predetermined pH level, after a predetermined time has elapsed, or if a predetermined biomarker or one or more physiological compounds are present within the surrounding environment.

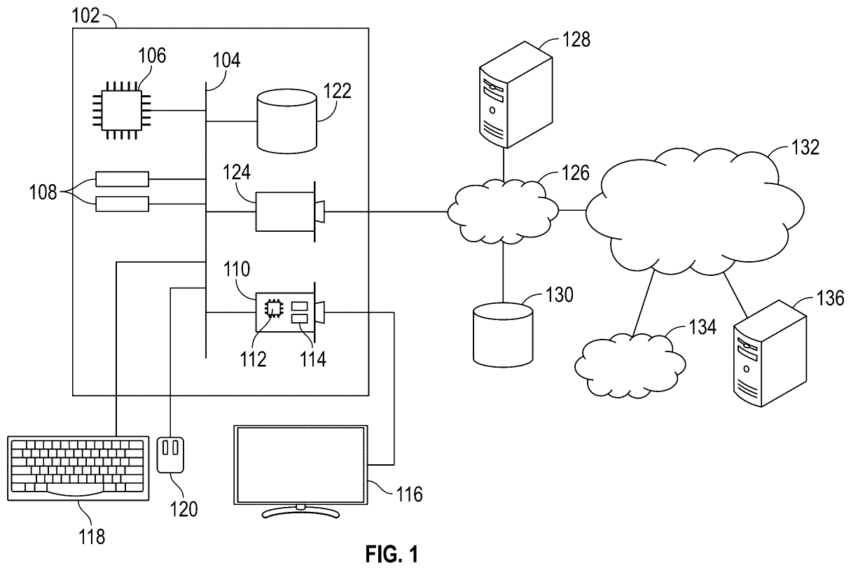

FIG. 1 illustrates an exemplary hardware platform in accordance with embodiments described herein. Computer 102 can be a desktop computer, a laptop computer, a server computer, a mobile device such as a smartphone or tablet, or any other form factor of general or special-purpose computing device. Depicted with computer 102 are several components for illustrative purposes. In some embodiments, certain components may be arranged differently or absent. Additional components may also be present. Included in computer 102 is system bus 104, whereby other components of computer 102 can communicate with each other. In certain embodiments, there may be multiple buses, or components may communicate with each other directly. Connected to system bus 104 is central processing unit 106, also known as a CPU. Also attached to system bus 104 are one or more random-access memory (RAM) modules 108. Also attached to system bus 104 is graphics card 110. In some embodiments, graphics card 110 may not be a physically separate card but may be integrated into the motherboard or the central processing unit 106. In some embodiments, graphics card 110 has a separate graphics-processing unit (GPU) 112, which can be used for graphics processing or general-purpose computing (GPGPU). Also on graphics card 110 is GPU memory 114. Connected (directly or indirectly) to graphics card 110 is display 116 for user interaction. In some embodiments, no display is present, while in others, it is integrated into computer 102. Similarly, peripherals such as keyboard 118 and mouse 120 are connected to system bus 104. Like display 116, these peripherals may be integrated into computer 102 or absent. Also connected to system bus 104 is local storage 122, which may be any form of computer-readable media and may be internally installed in computer 102 or externally and removably attached.

Such non-transitory computer-readable media include both volatile and nonvolatile media, removable and nonremovable media, and contemplate media readable by a database. For example, non-transitory computer-readable media include (but are not limited to) RAM, ROM, EEPROM, flash memory or other memory technology, CD-ROM, digital versatile discs (DVD), holographic media or other optical disc storage, magnetic cassettes, magnetic tape, magnetic disk storage, and other magnetic storage devices. These technologies can store data temporarily or permanently. However, unless explicitly specified otherwise, the term “computer-readable media” should not be construed to include physical but transitory forms of signal transmission such as radio broadcasts, electrical signals through a wire, or light pulses through a fiber-optic cable. Examples of stored information include computer-executable instructions (for example, non-transitory computer-executable instructions that, when executed by a processor, perform the methods disclosed herein), data structures, program modules, and other data representations.

Finally, network interface card 124 (also known as a NIC) is attached to system bus 104 and allows computer 102 to communicate over a network such as local network 126. Network interface card 124 can be any form of network interface known in the art, such as Ethernet, ATM, fiber, Bluetooth®, or Wi-Fi (i.e., the IEEE 802.11 family of standards). Network interface card 124 connects computer 102 to local network 126, which may include one or more other computers, such as computer 128, and network storage, such as data store 130. Generally, a data store such as data store 130 may be any repository from which information can be stored and retrieved as needed. Examples of data stores include relational or object-oriented databases, spreadsheets, file systems, flat files, directory services such as LDAP and Active Directory, or email storage systems. A data store may be accessible via a complex API (such as, for example, Structured Query Language), a simple API that provides only read, write, and seek operations, or any level of complexity in between. Some data stores may additionally provide management functions for data sets stored therein, such as backup or versioning. Data stores can be local to a single computer, such as computer 128, accessible on a local network, such as local network 126, or remotely accessible over Internet 132. Local network 126 is, in turn, connected to Internet 132, which connects many networks such as local network 126, remote network 134, or directly attached computers such as computer 136. In some embodiments, computer 102 can itself be directly connected to Internet 132.

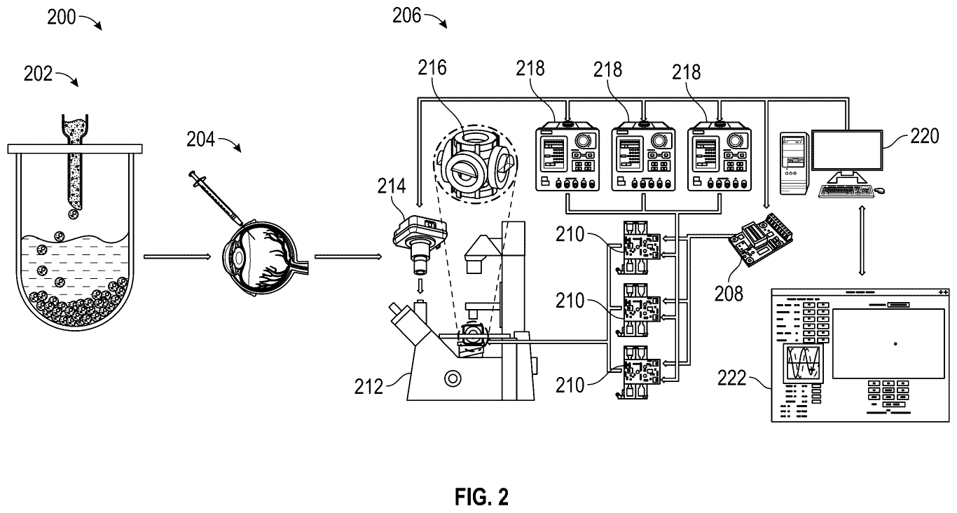

FIG. 2 illustrates an exemplary drug delivery system 200 in accordance with embodiments described herein. For example, exemplary drug delivery system 200 includes administering (e.g., injecting) a microbot, fabricated using a fabrication method 202, into an ocular environment 204. Exemplary drug delivery system 200 further includes a magnetic control system (MCS) 206, where the microbot is navigated within a biofluid of ocular environment 204 using MCS 206. MCS 206 may comprise one or more signal generators 208, one or more drive circuits 210, one or more microscopic components 212, one or more cameras 214, one or more magnetic coils 216, one or more power supplies 218, a computer 220, and an interface 222.

In some embodiments, MCS 206 is configured to generate magnetic fields to control the movement of the microbot (e.g., controlling the propulsion of the microbot within the biofluid). MCS 206 may comprise a magnetic system, which includes one or more signal generators 208, drive circuits 210, magnetic coils 216, and power supply 218. For example, magnetic coils 216 may include one or more Helmholtz coils. For instance, magnetic coils 216 may include triaxial Helmholtz coils. The triaxial Helmholtz coils (e.g., three pairs of Helmholtz coils) may be arranged along an X-axis, a Y-axis, and a Z-axis to produce a three-dimensional (3D) uniform magnetic field, allowing for control of the generated magnetic field along all three axes. Magnetic coils 216 may be used to create static or dynamic fields with different orientations and magnitudes for controlling the microbot's movement (e.g., propulsion) within the biofluid. In some embodiments, the magnetic fields generated by MCS 206 are uniform. In other embodiments, the magnetic fields generated by MCS 206 are non-uniform, where magnetic field gradients are present. The magnetic system may produce rotating, oscillating, or uniform fields to propel particles in specific trajectories by adjusting the parameters of the magnetic fields.

In some embodiments, the magnetic fields generated by MCS 206 may be described by the following equation:

$$\vec{B} = [ \begin{matrix}{B}_{s} cos θ + {B}_{r} sin θcosω t \\ {B}_{s} sin θ + {B}_{r} cos θcosω t \\ {B}_{r} sin ω t\end{matrix} ] ,$$

$$\vec{n} = [ \begin{matrix}cos θ & sin θ & 0\end{matrix} ] ,$$

where Bₛ is the static magnetic field amplitude, Bᵣ is the rotational magnetic field amplitude, ω is the rotational frequency (rad/s), t is the time (seconds), and θ is the heading angle of the propulsion direction of the microbot within the biofluid. In some embodiments, Bᵣ may be adjusted to be proportional to the applied frequency (|Bᵣ|=0.5f, where f is the frequency in Hz) in order to prevent the magnetic rotation of the microbot from desynchronizing with the rotation frequency of the magnetic field generated by MCS 206. In some embodiments, the propulsion direction of the microbot within the biofluid may be defined to be along a rotation axis and may correspond to a heading vector (\vec{n}). When looking from behind the heading vector (\vec{n}), the rotation of the microbot may be defined as counterclockwise with positive ω and clockwise with negative ω. In a Newtonian fluid (e.g., water), when there is no static field (Bₛ=0) and the rotational frequency is below a critical step-out frequency, a microbot with a permanent dipole moment may rotate about the heading vector (\vec{n}) at the same rotational frequency as the field, where the dipole moment of the microbot may be in the plane perpendicular to n. When there is an additional static field, the dipole moment of the microbot may be tilted at a constant angle out of the plane perpendicular to \vec{n}, and the dipole moment and microbot may rotate about \vec{n} at the same frequency as the field, which makes the dipole moment of the microbot co-rotate with the field.

In some embodiments, one or more signal generators 208 may provide an input electrical signal that determines how the current behaves in magnetic coils 216. For example, one or more signal generators 208 may be configured to set the alternating current (AC) frequency, which may determine how quickly the generated magnetic field oscillate. In another example, one or more signal generators 208 may be configured to regulate the strength of the current flowing through magnetic coils 216, which may directly influence the magnitude of the generated magnetic field. For instance, a DC signal from one or more signal generators 208 may create a static magnetic field, where drive circuits 210 actively switch the direction of the current flowing through magnetic coils 216. In contrast, an AC signal from one or more signal generators 208 may create an oscillating magnetic field at a desired frequency and amplitude.

In some embodiments, drive circuits 210 may be configured to adjust the direction of current flowing through magnetic coils 216. In some embodiments, drive circuits 210 are an H-bridge and/or a bridge-tied load amplifier. By switching the direction of the current flowing through magnetic coils 216, drive circuits 210 may create a magnetic field that alternates directions. Further, drive circuits may use pulse-width modulation (PWM) to adjust the effective current flowing through magnetic coils 216 for fine-tuning the strength of the generated magnetic field. For example, if the one or more signal generators 208 produce a DC signal, drive circuits 210 may ensure that the DC signal is delivered symmetrically to magnetic coils 216. Using one or more signal generators 208 and drive circuits 210 together enables fine control over the parameters of the magnetic field generated by MCS 206. For example, the field strength of the magnetic field may be adjusted by increasing or decreasing the amplitude of the signal being received from the one or more signal generators 208.

In some embodiments, each magnetic coil 216 is electrically coupled to a separate power supply 218. In other embodiments, single power supply 218 is electrically coupled to all magnetic coils 216 used at a given time. Power supply 218 provides the necessary voltage and current to MCS 206 for generating the magnetic field. For example, power supply 218 may ensure that a desired voltage is provided to drive circuits 210 and magnetic coils 216. In another example, power supply 218 may ensure that a required current is provided to magnetic coils 216 so magnetic coils 216 produce a magnetic field with a desired field strength. For example, the applied magnetic field may cause the microbot to roll and generate a force caused by the applied magnetic field.

The applied magnetic field may cause the microbot to move by magnetic actuation. For example, the applied magnetic field may cause the microbot to roll, spin, or change orientation based on the direction and strength of the magnetic field. The force generated may perform mechanical work, such as moving the microbot through a fluid. The mechanical work may be a movement of one or more portions of the microbot.

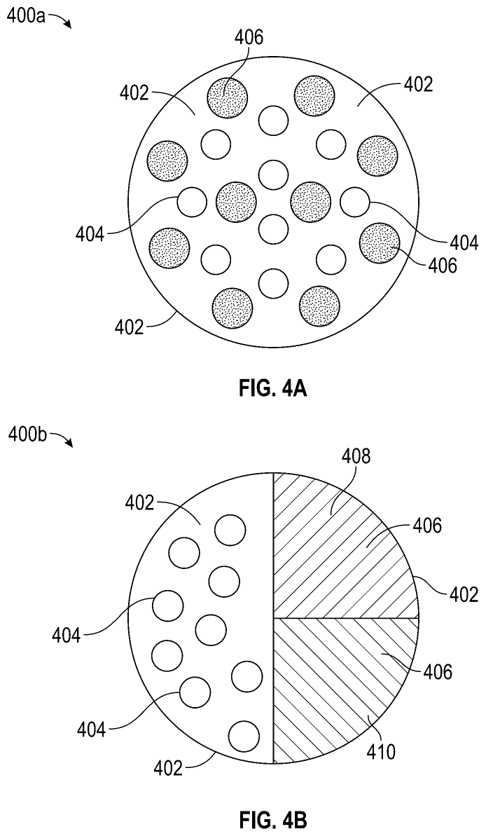

In some embodiments, the microbot is selectively propelled within the biofluid based on selective rotation and field strength of the magnetic field generated by MCS 206. In some embodiments, a plurality of microbots is selectively propelled within the biofluid by MCS 206 at the same time. Magnetic coils 216 may be positioned so the microbot, which may be located in an ocular environment of a living organism, may be housed within (e.g., surrounded by) and affected by the magnetic field generated by MCS 206. As further discussed below in FIGS. 4A-4B, the microbot may comprise magnetic particles (e.g., ferromagnetic or paramagnetic particles). The magnetic particles dispersed throughout the microbot may experience a force and/or torque when subjected to the external magnetic field generated by MCS 206, causing the microbot to align and/or react (e.g., propel) to the field direction of the magnetic field. For example, the magnetic propulsion of the microbot may cause the microbot to roll (e.g., rotate) throughout the biofluid by generating a force caused by the applied magnetic field.

MCS 206 may further comprise microscopic component 212, camera 214, computer 220, and interface 222 for providing computer-controlled propulsion and navigation of the microbot. MCS 206 may be executed on any computer system now known or later developed, including, but not limited to, those discussed above in FIG. 1.

Computer 220 may be communicatively coupled to interface 222 to enable visualization and manipulation of the microbot suspended in the biofluid. For example, computer 220 may serve as the central hub, running control software that interfaces with both the magnetic system and an imaging system of MCS 206. Computer 220 may connect to interface 222 (e.g., a data acquisition device, microcontroller, or FPGA board) via USB, Ethernet, or wireless protocols. Interface 222 may translate the digital commands from computer 220 into analog signals or PWM signals, which may be sent to the magnetic system. These signals may adjust parameters, such as the magnetic field's strength, direction, frequency, and/or waveform.

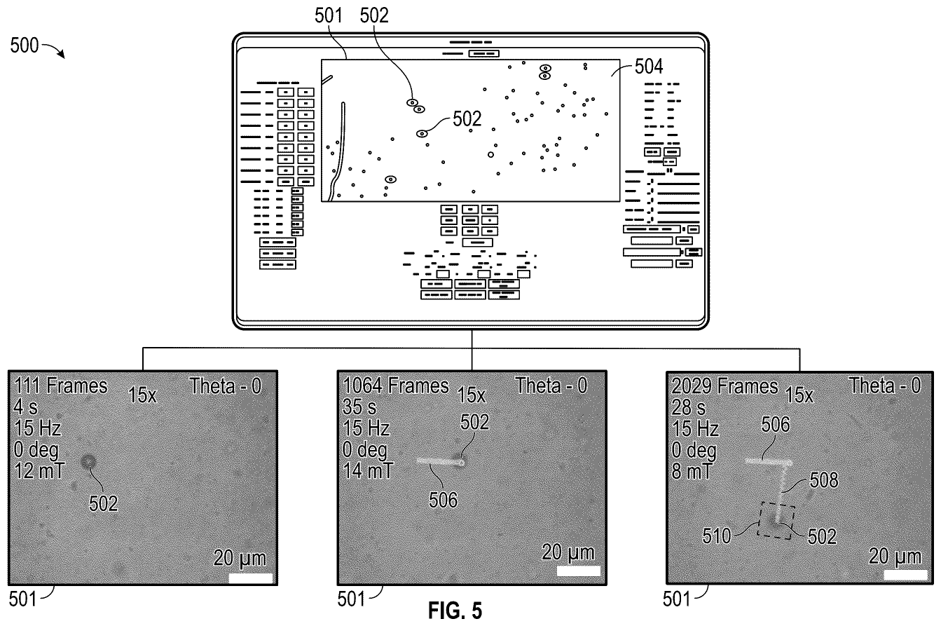

Interface 222 may be configured for adjusting the magnetic field parameters of MCS 206 so the microbot may be propelled in a desired direction and to a desired location within the biofluid (e.g., to the target ocular region). Using interface 222 to visualize and control the microbot within the biofluid is further discussed below in FIG. 5.

In some embodiments, the visualization of the microbot is provided by an imaging system that includes microscopic component 212 and camera 214. To capture and visualize the motion of the microbot, MCS 206 may integrate a microscopic element, such as a microscope or a stereoscopic camera, for providing a visual of the microbot. For example, one or more microscopic components 212 may magnify a region of interest within the biofluid, while camera 214 records the motion of the microbot in real-time. Visual data of the microbot may then be instantaneously transmitted to computer 220 and displayed on interface 222.

In some embodiments, the visual data may be combined with feedback algorithms so MCS 206 may support closed-loop control. For example, if camera 214 detects deviations in the trajectory of the microbot from the desired path, the software may adjust various parameters, via interface 222, of the magnetic field strength or direction to correct the movement of the microbot. In some embodiments, a user may manually adjust the parameters via interface 222. Together, the magnetic system and the imaging system of MCS 206 facilitates real-time control and monitoring of the behavior of the microbot, allowing users to adjust the parameters of MCS 206 to propel the microbot to desired directions at desired velocities within the biofluid.

In some embodiments, MCS 206 may be a portable device readily deployed in hospitals or field medical stations. For example, the portable system may contain the magnetic system and the imaging system of MCS 206 such that the portable system contains magnetic coils capable of housing a full-sized human head, provides visualization inside the eye through a stereoscopic camera, and is equipped with a portable laptop computer with software interfacing for direct and automatic microbot control via interface 222.

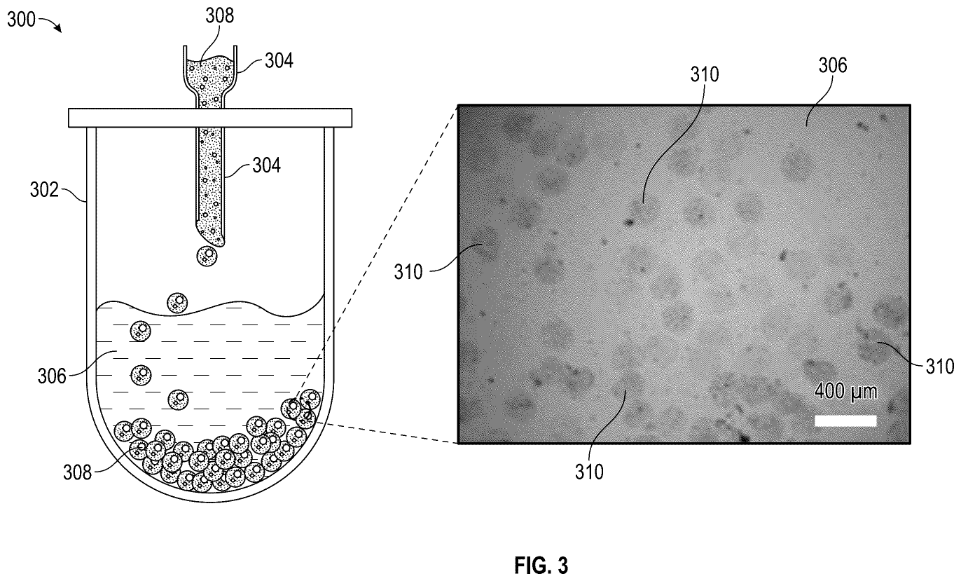

FIG. 3 illustrates an exemplary fabrication method 300 for fabricating microbots in accordance with embodiments described herein. Fabrication method 300 may include a centrifuge tube 302, a needle 304, an aqueous solution 306, a microbot mixture 308, and one or more microbots 310.

Fabrication method 300 involves the production of microbots 310 using a centrifuge-based approach. The fabrication method 300 begins by placing the centrifuge tube 302, containing the aqueous solution 306, into a centrifuge. The needle 304 is securely attached to a hole at the top of the centrifuge tube 302 for extrusion. The centrifuge applies a preset relative centrifugal force (RCF) to push droplets of the microbot mixture 308 through the needle 304 into the aqueous solution 306. As the droplets enter the aqueous solution 306, crosslinking occurs, solidifying them into microbots 310. The size of the resulting microbots 310 may be controlled by adjusting parameters such as the diameter of needle 304, the applied RCF, or the properties of the microbot mixture 308. The diameter of the microbots 310 may be estimated using the formula

$${d}_{p} = \sqrt[3]{( 6 {d}_{n} {σ}_{p} ) / ( {ρ}_{ℊ} ℊ )} ,$$

where dₚ is the diameter of the microbots 310, dₙ is the diameter of the needle 304, op is the surface tension of the biocompatible material, ρ_{g} is the density of the biocompatible material, and g is the applied RCF.

In some embodiments, a surface of centrifuge tube 302 contains a hole that needle 304 enters through. Needle 304 may be a medical needle or a non-medical needle. For example, needle 304 may be a microneedle or a hypodermic needle. Needle 304 may be loaded with microbot mixture 308. Microbot mixture 308 may include a drug payload, magnetic particles, biocompatible material, and/or chemical additives. The drug payload may include a variety of therapeutic or non-therapeutic agents, depending on the intended application. For example, the drug payload may include thrombolytic compounds, such as tissue plasminogen activator (tPA) or streptokinase, which are designed to dissolve blood clots and restore normal blood flow within a living organism. The drug payload may further include protein molecules, such as BDNF protein molecules, which promote neural survival and regeneration of neurons. The drug payload may also incorporate other drug types, such as small molecules, nucleic acids (e.g., siRNA or mRNA), or imaging agents for diagnostic purposes.

Microbot mixture 308 may include magnetic particles, such as paramagnetic iron oxide nanoparticles, or biocompatible material, such as an aqueous alginate solution (e.g., alginate hydrogel). For example, microbot mixture 308 may comprise a 0.5% concentration of an aqueous alginate solution and a 1% concentration of magnetic particles, such as iron (III) oxide magnetic nanoparticles. Microbot mixture 308 may further include chemical additives such as co-polymers. The chemical additives may also be considered part of the drug payload. In some embodiments, microbot mixture 308 may include any range of concentrations of any drug payload, magnetic particles, biocompatible material, or chemical additives.

As an illustrative example, fabrication method 300 may utilize a 1.5 mL centrifuge tube 302 with a hole at the top, sized for a 32/34-gauge needle to press-fit securely. The aqueous solution 306 may be a calcium chloride solution, into which the droplets of microbot mixture 308 are extruded. As the centrifuge spins the centrifuge tube 302 around a central axis, microbot mixture 308 is extruded through needle 304 into aqueous solution 306, where crosslinking occurs to solidify the droplets into microbots 310.

Thus, the droplets extruded from needle 304 during fabrication method 300 may form microbots 310, which may contain varying concentrations of biocompatible materials, magnetic particles, chemical additives, and/or a drug payload. In some embodiments, the concentration of the biocompatible material (e.g., sodium alginate) in microbot mixture 308 may be adjusted so the chemical additives modulate the shrinking or swelling properties of microbots 310 in a desired manner. Fabrication method 300 may allow tens to thousands of microbots 310 to be produced in a matter of seconds.

FIGS. 4A-4B illustrate exemplary microbots 400a-b used in accordance with embodiments described herein. Microbots 400 may be fabricated by, but is not limited to, fabrication method 300 discussed above in FIG. 3. Microbots 400 may include all features and aspects of the microbots discussed in FIG. 3. In some embodiments, microbots 400 may comprise a biocompatible body 402, magnetic particles 404, and a drug payload 406. Microbots 400 may be any shape or size. In some embodiments, microbots 400 are a spherical shape. In some embodiments, microbots 400 may have a width ranging between 1 μm to 200 μm.

In some embodiments, biocompatible body 402 is a biocompatible material. For example, biocompatible body 402 may be any material formed from crosslinking (which induces gel formation) the biocompatible material and aqueous solution 306 discussed above in FIG. 3. For instance, biocompatible body 402 may be an alginate hydrogel composed of sodium alginate, calcium ions, and/or water. In some embodiments, biocompatible body 402 may be any alginate crosslinked with divalent cations used to form an alginate hydrogel. In some embodiments, biocompatible body 402 may include a polymer material, such as chitosan, collagen, gelatin, or fibrin. In other embodiments, biocompatible body 402 may be a synthetic polymer. In some embodiments, an external surface of biocompatible body 402 may be coated with biomolecules such as avidin, biotin, biotin-PEG3-Amine, or chitosan biotin.

In some embodiments, biocompatible body 402 may comprise a composite or hybrid hydrogel formed from a combination of natural polymers and synthetic polymers, including stimuli-responsive polymers configured to modulate swelling, shrinking, or drug payload 406 release in response to temperature or other environmental conditions. For example, biocompatible body 402 may comprise a composite hydrogel including sodium alginate and a thermo-responsive polymer, such as poly (N-isopropylacrylamide) (PNIPAM).

In some embodiments, the composite hydrogel may comprise about 0.5-5 weight percentage (wt %) of sodium alginate and about 0.1-5 wt % of PNIPAM, although other concentrations are contemplated and may be selected based on desired mechanical, swelling, or release properties. In some embodiments, PNIPAM may be grafted, polymerized, or otherwise bonded to the sodium alginate using one or more chemical crosslinkers, crosslinker initiators, and/or crosslinker accelerators. In some embodiments, the resulting mixture may undergo ultraviolet light curing, heating, or other polymerization conditions to form a polymerized hydrogel. In some embodiments, the crosslinker concentration may range from about 0.01-1 wt %, and the crosslinker initiator or crosslinker accelerator concentration may range from about 0.01-1 wt %, although such values are not limiting. For example, the crosslinker concentration, crosslinker initiator, and/or the crosslinker accelerator concentration may range from about 0.01-0.1 wt %, 0.1-0.2 wt %, 0.2-0.3 wt %, 0.3-0.4 wt %, 0.4-0.5 wt %, 0.5-0.6 wt %, 0.6-0.7 wt %, 0.7-0.8 wt %, 0.8-0.9 wt %, or 0.9-1 wt %. In some embodiments, the resulting hydrogel may be mechanically processed, sonicated, homogenized, or otherwise treated to adjust particle size and uniformity.

In some embodiments, magnetic particles 404 may include any ferromagnetic or paramagnetic particles. For example, magnetic particles 404 may include iron (III) oxide nanoparticles. Magnetic particles 404 enable external magnetic control of microbots 400. For example, MCS 206, as discussed in FIG. 2, may facilitate the navigation of microbots 400 to target regions within a biofluid using visual and/or non-visual feedback.

In some embodiments, drug payload 406 may include any therapeutic or non-therapeutic agents discussed in FIG. 3 above. For example, drug payload 406 may include thrombolytic compounds, such as tPA or streptokinase. Drug payload 406 may further include protein molecules (e.g., neuroprotective or anti-inflammatory proteins) such as BDNF protein molecules, which promote neural survival and regeneration of neurons. The drug payload may also incorporate other drug types, such as small molecules, nucleic acids (e.g., siRNA or mRNA), or imaging agents for diagnostic purposes.

In some embodiments, drug payload 406 may include chemical additives such as co-polymers. The chemical additives may allow for selective modulation of a release rate (e.g., diffusion) of drug payload 406 from biocompatible body 402. In some embodiments, when a predetermined condition is met, microbots 400 may expand and release drug payload 406 into a surrounding environment. For example, microbots 400 may be configured to automatically release drug payload 406 into the surrounding environment of a target ocular region in response to the condition being met. For instance, microbots 400 may be chemically modified with co-polymer additives to respond to temperature changes in the surrounding environment of the biofluid. At a lower temperature (e.g., room temperature), microbots 400 may be designed to retain drug payload 406. However, when the surrounding environment reaches a predetermined temperature (e.g., normal body temperature), the co-polymer additives may cause microbots 400 to expand and release drug payload 406.

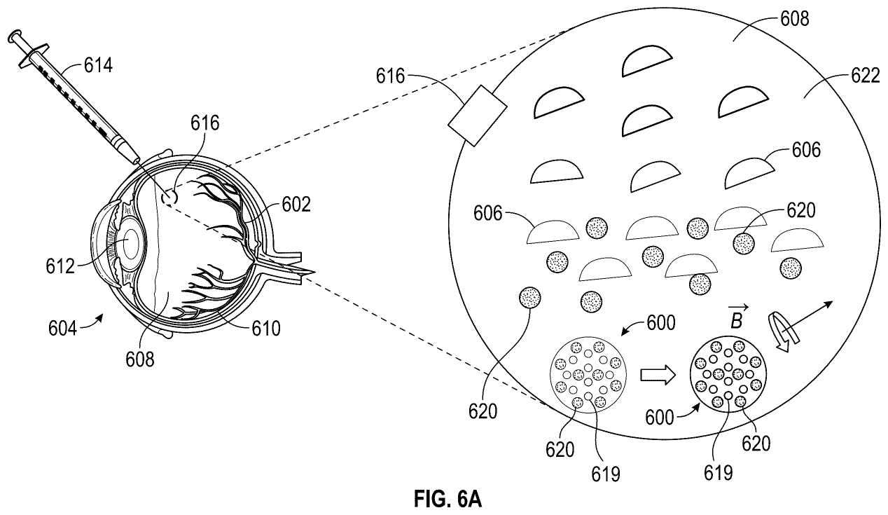

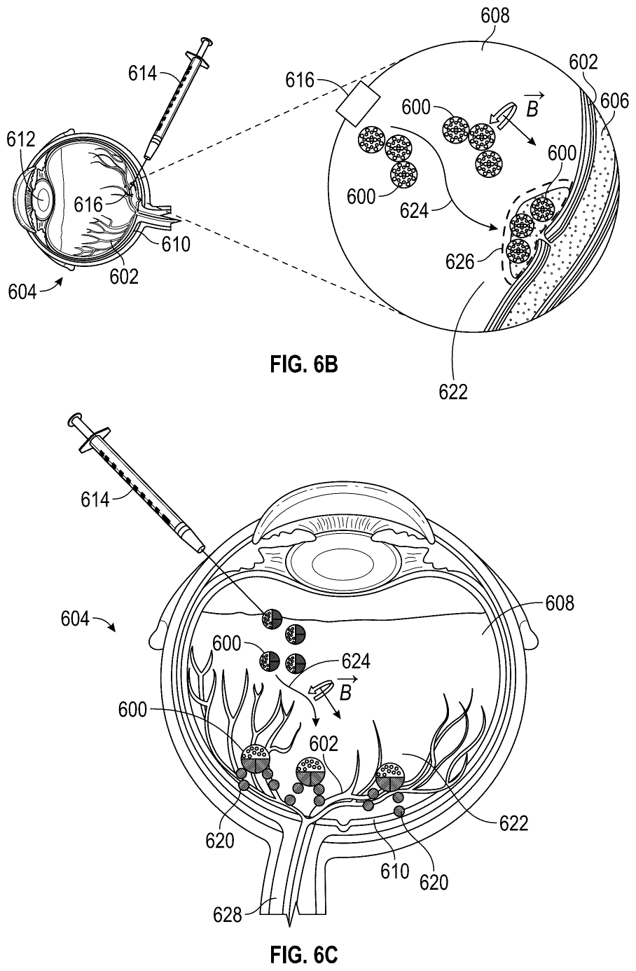

For example, the predetermined temperature may correspond to a critical solution temperature of one or more components of the biocompatible body 402. For instance, the predetermined temperature may be about 28° C., although it is contemplated herein that any temperature may be used in accordance with the embodiments disclosed herein to selectively modulate the release of the drug payload 406 within a biofluid. For example, in some embodiments, the predetermined temperature may be selected from a range of about 10-20° C., 20-25° C., 25-30° C., 30-35° C., 35-40° C. or 40-45° C. In some embodiments, a portion of the biocompatible body 402 may be soluble in the biofluid at temperatures below the predetermined temperature and become partially or fully insoluble when the surrounding environment (e.g., surrounding environment 622 in reference to FIGS. 6A-6C) exceeds the predetermined temperature, thereby triggering expansion of the microbots 400 and release of the drug payload 406. In such embodiments, release of the drug payload 406 may occur when the surrounding environment transitions above the critical solution temperature.

In some embodiments, the release rate of drug payload 406 is a controlled rate so the drug payload is uniformly released over a predetermined length of time. In some embodiments, the concentration of biocompatible body 402, such as sodium alginate, may be adjusted and combined with copolymers to allow for selective modulation of the shrinking and swelling properties of microbots 400 for regulating how fast or slow drug payload 406 diffuses out of microbots 400. In some embodiments, the condition that may be met for microbots 400 to expand and release drug payload 406 includes the surrounding environment reaching the predetermined temperature, the surrounding environment reaching a predetermined pH level, or after a predetermined period of time has elapsed since the injection of microbots 400 into the biofluid.

In some embodiments, the release rate of drug payload 406 may be based on a pre-insertion temperature of microbots 400 prior to introduction into the biofluid. For example, cooling or heating microbots 400 prior to insertion into the biofluid may alter the physical state of the biocompatible body 402 and thereby influence subsequent swelling behavior and drug payload 406 release kinetics upon exposure to physiological conditions. For example, microbots 400 may be subjected to, heating, drying (e.g., air-drying), cooling, or freeze-drying processes prior to insertion, after which the microbots 400 may be configured to rehydrate, shrink, and/or re-expand upon contact with the biofluid or occurrence of a predetermined condition being met (e.g., predetermined temperature, predetermined pH level, after a predetermined time has elapsed, or if a predetermined biomarker or one or more physiological compounds are present within the surrounding environment of the biofluid), resulting in a modified release rate. In some embodiments, the release rate and duration of drug payload 406 may be selectively adjusted based on the thermal history, hydration state, or pre-conditioning of the microbots 400 prior to insertion.

In some embodiments, release of drug payload 406 from microbots 400 may be triggered in response to multiple activation mechanisms, including but not limited to exposure to chemical agents, biological agents, pathogens, toxins, or other changes in physiological or environmental conditions. In some embodiments, the microbots 400 may be configured to respond to chemical inhalation, dermal exposure, or changes in concentrations of one or more compounds present in the biofluid or surrounding environment, thereby initiating payload release.

In some embodiments, microbots 400 may further include sensing or indicator functionalities, such that exposure to an agent of interest causes the microbots 400 to change optical properties, emit detectable signals, or release secondary payloads. For example, the microbots 400 may be configured to change color, emit fluorescence, release aromas, or other detectable markers, or otherwise provide a collective visual, chemical, or sensory indication of exposure.

In some embodiments, microbots 400 may be applied to or located at a surface of an object or body (e.g., skin), including being applied via a topical formulation, coating, cream, or lotion, such that activation of the microbots 400 provides localized or systemic situational awareness, health monitoring, or exposure detection. In such embodiments, information generated by the microbots 400 may be used for visual detection, physiological monitoring, or data integration with external monitoring systems.

Magnetic particles 404 and drug payload 406 may be embedded, encapsulated, or dispersed throughout biocompatible body 402 in any manner. For example, magnetic particles 404 and the compounds that make up drug payload 406 may be randomly distributed throughout biocompatible body 402. In some embodiments, drug payload 406 may be located within a predetermined section of biocompatible body 402, while magnetic particles 404 may be located in a different section of biocompatible body 402. As an illustrative example, microbot 400a may comprise magnetic particles 404 and drug payload 406 dispersed randomly throughout biocompatible body 402. As another illustrative example, microbot 400b may comprise magnetic particles 404 and drug payload 406 disposed in a non-random distribution. For example, a first section 408 of microbots 400a may comprise a first type of drug payload material, such as a neuroprotective protein. In contrast, a second section 410 may comprise a second type of drug payload material, such as an anti-inflammatory protein.

FIG. 5 illustrates an exemplary interface 500 used in accordance with embodiments described herein. Interface 500 may include all aspects and features of interface 222 disclosed above in FIG. 2.

In some embodiments, interface 500 may overlay a visual feed 501 with tracking algorithms that highlight paths, velocity, or directional changes of one or more microbots 502 within a biofluid 504. In some embodiments, interface 500 allows a user to monitor and adjust field parameters of magnetic fields (such as the magnetic field generated by MCS 206 disclosed in FIG. 2) in real time. For example, interface 500 may adjust parameters such as a magnetic field's strength, direction, frequency, and waveform. Control over these parameters enables the propulsion of microbots 502 at various speeds and directions. For example, interface 500 may be used to navigate microbots 502 in a first direction 506 and a second direction 508 to a target region 510.

Microbots 502 may be navigated in any direction via interface 500. For example, microbots 502 may be configured to be propelled within the biofluid 504 via interface 500 along any of an x-axis, a y-axis, or a z-axis. The magnetic navigation of microbots 502 may be open-loop or closed-loop. For example, closed-loop navigation of microbots 502 may include microbots 502 being navigated along pre-planned trajectories to a target region without user input. In contrast, open-loop navigation may involve applying a fixed magnetic field or pre-determined magnetic field patterns to guide the microbots 502, without real-time feedback or adjustments during the movement of microbots 502. In some embodiments, if tracking of microbots 502 is lost or degraded, dynamic modeling techniques (e.g., machine learning models, deep neural networks (DNNs), and Kalman filters) may be employed to estimate the position, trajectory, or state of microbots 502, thereby allowing navigation of microbots 502 to continue in the absence of real-time positional feedback. In some embodiments, the dynamic modeling techniques may be used generally for the tracking and navigation of microbots 502 discussed herein and are not limited to situations of lost or degraded tracking.

In some embodiments, interface 500 may display live readouts of the magnetic field strength at microbots' 502 location. In some embodiments, interface 500 may provide real-time metrics regarding microbots 502, such as velocity, displacement, and direction of movement.

To ensure microbots 502 behave appropriately within the biofluid 504, interface 500 may display metrics of the surrounding environment of microbots 502. For example, sensors or integrated feedback systems may measure and display parameters such as pH, temperature, or fluid viscosity in real-time. In some embodiments, visual indicators or warnings may be displayed by interface 500 to notify the user if conditions deviate from acceptable ranges, enabling rapid adjustments to the navigation strategy. Further, interface 500 may display alerts to alert the user to a collision, or potential collision, between microbots 502 and obstacles within the biofluid 504, such as tissues, cellular structures, or synthetic barriers. For example, alerts may be triggered when a microbot 502 is within a predefined proximity to an obstacle, prompting the user to intervene or allowing the system to autonomously adjust the magnetic field to safely redirect the microbot 502. In some embodiments, interface 500 may employ advanced image processing and machine learning algorithms to detect potential obstacles in the path of microbots 502.

In some embodiments, interface 500 may facilitate manual or automated navigation of microbots 502 to establish contact with target objects within target region 510. For example, a user may navigate microbots 502 via interface 500 so microbots 502 contact red blood cells in a target region 510. Once contact has been made, a drug payload may then diffuse from microbots 502 to break down coagulated blood caused by, for example, a vitreous hemorrhage.

In some embodiments, microbots 502 are magnetically removed from biofluid 504. For example, microbots 502 may be extracted using a magnetized needle (such as needle 304 discussed above in FIG. 3). For instance, the magnetized needle may localize and capture microbots 502 based on the magnetic attraction of microbots 502 to generated magnetic fields.

Alternatively, electrostatic methods may be employed, where microbots 502 may be attracted to a charged needle or electrode, facilitating the extraction of microbots 502. In some embodiments, microbots 502 may be navigated to a predetermined extraction point using interface 500 to position microbots 502 proximal to the magnetized needle, or other needle, for facilitating the extraction of microbots 502. In some embodiments, interior surfaces of the syringe and/or magnetic needle used during the extraction process may be coated with a surfactant to reduce adhesion between microbots 502 and inner walls of the magnetic needle and to facilitate smooth passage of microbots 502 through the syringe and/or needle.

In some embodiments, the localization and control of microbots 502 discussed herein may be performed without optical or imaging-based feedback (non-vision-based localization), including using magnetic field measurements, predictive models, or machine learning-based estimators in combination with a feedback controller.

In some embodiments, outputs of the machine learning model may be combined with a dynamic or predictive model using a state estimation technique, such as a Kalman filter, to combine measurement data and model predictions for improved localization accuracy of microbots 502. This non-vision-based approach may be advantageous in situations where optical or imaging conditions are compromised, including conditions associated with corneal opacity, intraocular bleeding, clouded biofluids, tissue damage, inflammation, or other obstructions that limit visibility within an ocular environment.

In some embodiments, the machine learning modeling and predictive modeling techniques discussed herein may further be used in combination with one or more components of MCS 206, referenced above in FIG. 2, for the localization and control of microbots 502. For example, the machine learning modeling and predictive modeling techniques are not limited to non-visual-based situations and may be used in combination with one or more cameras (e.g., camera 214 referenced above in FIG. 2) providing a visual indicator of microbots 502.

FIG. 6A illustrates a first exemplary depiction of treating an ocular injury using microbots 600. Microbots 600 may have all aspects and features of microbots disclosed in FIGS. 2-5. In some embodiments, microbots 600 may be used to treat a vitreous hemorrhage injury, where a blood vessel 602 in an eye 604 has been ruptured, causing red blood cells 606 to pool together. For example, red blood cells 606 may pool together in an ocular component, such as in the vitreous humor 608. The vitreous humor 608 is located between a retina 610 and a lens 612 of the eye 604.

In some embodiments, microbots 600 are administered into eye 604 at an administering site 616 using an administering device 614 (e.g., hypodermic needle). Administering site 616 may be a location on a surface of eye 604 so microbots 600 are administered into vitreous humor 608 upon injection. Any number (e.g., tens, hundreds, or thousands) of microbots 600 may be administered into vitreous humor 608 at one time. In some embodiments, microbots 600 may be in a pre-shaped formation before being administered. For example, a plurality of microbots 600 (e.g., a swarm) may be in a selective formation as microbots 600 are navigated through the vitreous humor 608 to the target ocular region.

Once microbots 600 are located within vitreous humor 608, microbots 600 may be navigated within a biofluid. Vitreous humor 608 may be comprised of the biofluid, wherein the biofluid may be any liquid in the body of a living organism through which particles (e.g., microbots 600, drug carriers, nanoparticles, or diagnostic agents) may be transported or directed for therapeutic or diagnostic purposes. For example, biofluid may be blood, lymph, cerebrospinal fluid, synovial fluid, interstitial fluid, or gastrointestinal fluid. For instance, once microbots 600 are located within vitreous humor 608, an external magnetic control system, such as MCS 206 discussed above in FIG. 2, may navigate microbots 600 to a target ocular region of vitreous humor 608. For example, the magnetic control system may be magnetically coupled to magnetic particles 619 comprised by microbots 600, where rotating magnetic fields may propel microbots 600 within vitreous humor 618. In some embodiments, a target ocular region may be a predetermined region within vitreous humor 608 with a concentration of red blood cells 606 above or below a minimum threshold. In some embodiments, the magnetic control system determines (e.g., detects) the target ocular region.

In some embodiments, once microbots 600 have been navigated to the target ocular region, microbots 600 may be configured to automatically release a drug payload 620 into a surrounding environment 622 of the target ocular region. In some embodiments, microbots 600 may not release drug payload 620 until a condition has been met. For example, microbots 600 may not release drug payload 620 until surrounding environment 622 (e.g., the biofluid) has reached a predetermined temperature. For instance, upon surrounding environment 622 reaching a predetermined temperature point, microbots 600 may release thrombolytic compounds, such as tPA or streptokinase. The thrombolytic compounds may then contact and degrade red blood cells 606 so red blood cells 606 dissolve at a faster rate than what would happen through natural recovery or existing surgical methods. In some embodiments, the released drug payload 620 causes the red blood cells 606 to degrade through hemolysis.

In some embodiments, microbots 600 are navigated to the target ocular region so microbots 600 physically contact red blood cells 606. For example, microbots 600 may forcefully hit and break up a plurality of red blood cells 606 before releasing drug payload 620. In some embodiments, while navigating through the surrounding environment 622, microbots 600 may engage, capture, or wrap one or more red blood cells 606 around themselves during movement, thereby facilitating mechanical disruption, transport, or enhanced therapeutic interaction with the plurality of red blood cells 606.

FIG. 6B illustrates a second exemplary depiction of treating an ocular injury using microbots 600. In some embodiments, microbots 600 are navigated along a surface of red blood cells 606 before releasing drug payload 620. For example, a pool of coagulated blood may form proximal to a ruptured blood vessel 602. Administered microbots 600 may be navigated via an external magnetic control system along a selective path 624 to a target ocular region 626. Microbots 600 may then navigate along the surface of red blood cells 606 within target ocular region 626 while simultaneously releasing a drug payload dispersed throughout microbots 600. In some embodiments, the magnetic propulsion of microbots 600 causes microbots 600 to roll (e.g., rotate) throughout the biofluid and along the surface of red blood cells 606 while releasing drug payload 620.

FIG. 6C illustrates a third exemplary depiction of treating an ocular injury using microbots 600. In some embodiments, microbots 600 may treat an ocular injury known as RGC loss. RGCs are neurons in the retina 610 that transmit visual information from eye 604 to the brain via the optic nerve 628. In some embodiments, microbots 600 are used to chemically alter intrinsic signaling near RGCs to promote the survivability of the RGCs and prevent the RGCs from dying. For example, microbots 600 may comprise drug payload 620 with certain therapeutic agents to promote the RGCs' survivability. For instance, drug payload 620 may comprise neuroprotective and/or anti-inflammatory proteins such as BDNF protein molecules.

Administered microbots 600 may be navigated via the external magnetic control system along a selective path 624 to a target ocular region 626. In some embodiments, once microbots 600 have been navigated to the target ocular region (e.g., a region within vitreous humor 608 proximal to retina 610), microbots 600 may be configured to automatically release drug payload 620 into surrounding environment 622 of the target ocular region 626. In some embodiments, microbots 600 may not release drug payload 620 until a condition has been met. For instance, upon surrounding environment 622 reaching a predetermined temperature point, microbots 600 may release BDNF protein molecules. The BDNF protein molecules may then be positioned near retina 610 to support the survivability of RGCs.

The microbots disclosed above in FIGS. 1-6 are not limited to treating injuries, such as blood clots, in an ocular region, and may be used in combination with a magnetic control system to treat similar injuries or conditions anywhere in a human body, such as in human veins, arteries, lungs, or hearts. For example, the microbots may be used for treating a blood clot in a deep vein (e.g., deep vein thrombosis) located in a patient's legs or pelvic area.

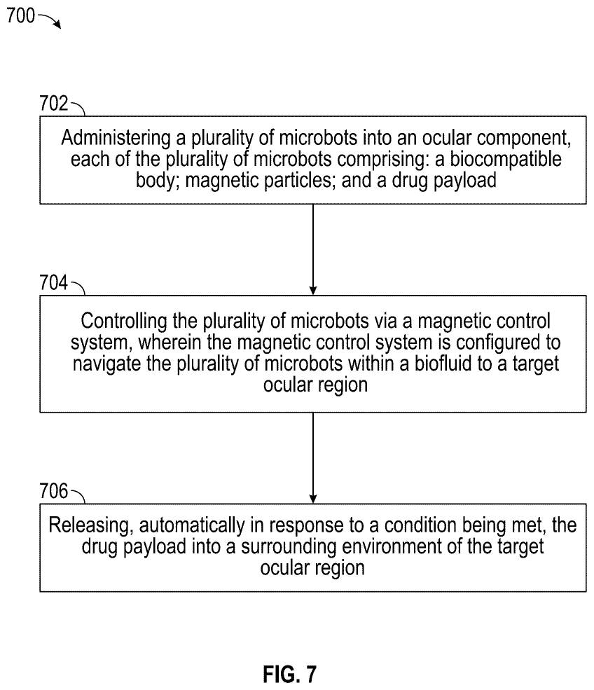

FIG. 7 illustrates an exemplary method 700 for delivering drugs to a target region in accordance with embodiments described herein. Method 700 may include all features and aspects of the microbots and magnetic control system described above in FIGS. 2-6B. At step 702, a plurality of microbots may be administered into an ocular component. Each of the plurality of microbots may comprise a biocompatible body, a plurality of magnetic particles, and a drug payload.

At step 704, the plurality of microbots may be controlled via a magnetic control system. The magnetic control system may be configured to navigate the plurality of microbots within a biofluid to a target ocular region.

At step 706, the drug payload may be released automatically, in response to a condition being met, into a surrounding environment of the target ocular region.

The following embodiments represent exemplary embodiments of concepts contemplated herein. Any one of the following embodiments may be combined in a multiple dependent manner to depend from one or more other clauses. Further, any combination of dependent embodiments (e.g., clauses that explicitly depend from a previous clause) may be combined while staying within the scope of aspects contemplated herein. The following clauses are exemplary in nature and are not limiting.
Clause 1. A microbot configured for delivering drugs to an ocular region, the microbot comprising: a biocompatible body; a plurality of magnetic particles; and a drug payload, wherein the microbot is configured to be navigated within a biofluid to a target ocular region via a magnetic control system, wherein the microbot is configured to automatically release the drug payload into a surrounding environment of the target ocular region in response to a condition being met.
Clause 2. The microbot of clause 1, wherein the drug payload comprises a thrombolytic compound configured to degrade coagulated blood upon the release of the drug payload into the surrounding environment.
Clause 3. The microbot of clauses 1 or 2, wherein the thrombolytic compound at least comprises tissue plasminogen activator (tPA) or streptokinase.
Clause 4. The microbot of any of clauses 1 through 3, wherein the drug payload comprises molecules configured to promote survivability of retinal ganglion cells (RGCs) upon the release of the drug payload into the surrounding environment.
Clause 5. The microbot of any of clauses 1 through 4, wherein the molecules are at least brain-derived neurotrophic factor (BDNF) protein molecules.
Clause 6. The microbot of any of clauses 1 through 5, wherein the condition is the surrounding environment reaching a predetermined temperature.
Clause 7. The microbot of any of clauses 1 through 6, wherein the drug payload comprises chemical additives configured to modulate a release rate of the release of the drug payload into the surrounding environment.
Clause 8. The microbot of any of clauses 1 through 7, wherein the chemical additives are co-polymer additives, wherein the release of the drug payload occurs upon the co-polymer additives detecting the predetermined temperature of the surrounding environment.
Clause 9. The microbot of any of clauses 1 through 8, wherein the release rate of the drug payload is a controlled rate such that the drug payload is uniformly released over a length of time upon the condition being met.
Clause 10. The microbot of any of clauses 1 through 9, wherein the condition is the surrounding environment reaching a predetermined pH level.
Clause 11. The microbot of any of clauses 1 through 10, wherein the plurality of magnetic particles and the drug payload are dispersed throughout the biocompatible body.
Clause 12. The microbot of any of clauses 1 through 11, wherein the biocompatible body is an alginate hydrogel.
Clause 13. The microbot of any of clauses 1 through 12, wherein the microbot has a width between 1 μm to 200 μm.
Clause 14. A system configured for delivering drugs to an ocular region, comprising: a microbot, comprising: a biocompatible body; a plurality of magnetic particles; and a drug payload; and a magnetic control system, wherein the magnetic control system is configured to navigate the microbot within a biofluid to a target ocular region, wherein the microbot is configured to automatically release the drug payload into a surrounding environment of the target ocular region in response to a condition being met.
Clause 15. The system of clause 14, wherein the magnetic control system comprises a plurality of Helmholtz coils, wherein selective rotation of the plurality of Helmholtz coils corresponds to selective movement of the microbot within the biofluid.
Clause 16. The system of clauses 14 or 15, wherein the magnetic control system is configured to navigate the microbot within the biofluid along any of an x-axis, a y-axis, or a z-axis.
Clause 17. The system of any of clauses 14 through 16, wherein the magnetic control system is configured to navigate the microbot to the target ocular region using closed-loop feedback.
Clause 18. The system of any of clauses 14 through 17, wherein the target ocular region is a location within a vitreous humor region.
Clause 19. The system of any of clauses 14 through 18, further comprising: a second microbot, comprising: a second biocompatible a second plurality of magnetic particles; and a second drug payload; and wherein the magnetic control system is configured to navigate the second microbot within the biofluid to the target ocular region, wherein the second microbot is configured to automatically release the second drug payload into the surrounding environment of the target ocular region in response to the condition being met.
Clause 20. A method for delivering drugs to an ocular region, the method comprising: administering a plurality of microbots into an ocular component, each of the plurality of microbots comprising: a biocompatible body; a plurality of magnetic particles; and a drug payload; controlling the plurality of microbots via a magnetic control system, wherein the magnetic control system is configured to navigate the plurality of microbots within a biofluid to a target ocular region; and releasing, automatically in response to a condition being met, the drug payload into a surrounding environment of the target ocular region.

Although the present disclosure has been described with reference to the embodiments illustrated in the attached drawing figures, it is noted that equivalents may be employed and substitutions made herein without departing from the scope of the present disclosure as recited in the claims.

Having thus described various embodiments of the present disclosure, what is claimed as new and desired to be protected by Letters Patent includes the following:

1. A microbot configured for delivering drugs to an ocular region, the microbot comprising:
a biocompatible body;
a plurality of magnetic particles dispersed throughout the biocompatible body; and
a drug payload dispersed throughout the biocompatible body,
wherein the microbot is configured to be navigated within a biofluid to a target ocular region via a magnetic control system,
wherein the microbot is configured to automatically release the drug payload into a surrounding environment of the target ocular region in response to a condition being met.

2. The microbot of claim 1, wherein the drug payload comprises a thrombolytic compound configured to degrade coagulated blood upon the release of the drug payload into the surrounding environment.

3. The microbot of claim 2, wherein the thrombolytic compound at least comprises tissue plasminogen activator (tPA) or streptokinase.

4. The microbot of claim 1, wherein the drug payload comprises molecules configured to promote survivability of retinal ganglion cells (RGCs) upon the release of the drug payload into the surrounding environment.

5. The microbot of claim 4, wherein the molecules are at least brain-derived neurotrophic factor (BDNF) protein molecules.

6. The microbot of claim 1, wherein the condition is the surrounding environment reaching a predetermined temperature.

7. The microbot of claim 6, wherein the drug payload comprises chemical additives configured to modulate a release rate of the release of the drug payload into the surrounding environment.

8. The microbot of claim 7, wherein the chemical additives are co-polymer additives, wherein the release of the drug payload occurs upon the co-polymer additives detecting the predetermined temperature of the surrounding environment.

9. The microbot of claim 7, wherein the release rate of the drug payload is a controlled rate such that the drug payload is uniformly released over a length of time upon the condition being met.

10. The microbot of claim 1, wherein the condition is the surrounding environment reaching a predetermined pH level.

11. The microbot of claim 1, wherein the plurality of magnetic particles and the drug payload are dispersed throughout the biocompatible body.

12. The microbot of claim 1, wherein the biocompatible body is an alginate hydrogel.

13. The microbot of claim 11, wherein the microbot has a width between 1 μm to 200 μm.

14. A system configured for delivering drugs to an ocular region, comprising:
a microbot, comprising:
a biocompatible body;
a plurality of magnetic particles dispersed throughout the biocompatible body;
a drug payload dispersed throughout the biocompatible body; and
a magnetic control system, wherein the magnetic control system is configured to navigate the microbot within a biofluid to a target ocular region,
wherein the microbot is configured to automatically release the drug payload into a surrounding environment of the target ocular region in response to a condition being met.

15. The system of claim 14, wherein the magnetic control system comprises a plurality of Helmholtz coils, wherein selective rotation of the plurality of Helmholtz coils corresponds to selective movement of the microbot within the biofluid.

16. The system of claim 15, wherein the magnetic control system is configured to navigate the microbot within the biofluid along any of an x-axis, a y-axis, or a z-axis.

17. The system of claim 16, wherein the magnetic control system is configured to navigate the microbot to the target ocular region using closed-loop feedback.

18. The system of claim 14, wherein the target ocular region is a location within a vitreous humor region.

19. The system of claim 14, further comprising:
a second microbot, comprising:
a second biocompatible body;
a second plurality of magnetic particles dispersed throughout the second biocompatible body; and
a second drug payload dispersed throughout the second biocompatible body,
wherein the magnetic control system is configured to navigate the second microbot within the biofluid to the target ocular region,
wherein the second microbot is configured to automatically release the second drug payload into the surrounding environment of the target ocular region in response to the condition being met.

20. A method for delivering drugs to an ocular region, the method comprising:
administering a plurality of microbots into an ocular component, each of the plurality of microbots comprising:
a biocompatible body;
a plurality of magnetic particles dispersed throughout the biocompatible body; and
a drug payload dispersed throughout the biocompatible body;
controlling the plurality of microbots via a magnetic control system, wherein the magnetic control system is configured to navigate the plurality of microbots within a biofluid to a target ocular region; and
releasing, automatically in response to a condition being met, the drug payload into a surrounding environment of the target ocular region.
